(19) United States
(12) Patent Application Publication
(10) Pub. No.: US 2015/0323530 A1
(43) Pub. Date: Nov. 12, 2015

(54) QUANTUM DOT-PROTEIN COMPLEXES, FILMS, AND METHODS OF USE

(71) Applicant: Jinye Wang, Shanghai (CN)

(72) Inventor: Jinye Wang, Shanghai (CN)

(73) Assignee: Empire Technology Development LLC, Wilmington, DE (US)

(21) Appl. No.: 14/410,524

(22) PCT Filed: Jul. 4, 2012
(86) PCT No.: PCT/CN2012/078168

§ 371 Date: Dec. 22, 2014

(51) Int. Cl. G01N 33/569; C07K 16/12; C07K 17/14; C07K 14/425

(52) U.S. Cl. CPC G01N 33/56911; C07K 14/425; C07K 16/12; C07K 17/14; G01N 2469/10

(57) ABSTRACT

The present application relates to protein-conjugated quantum dot compositions. The compositions may comprise, for example, zein proteins, and may be configured as films, for example on the sample-contacting surface of a sample well. Methods, kits, and an apparatus for the detection of food-borne microorganisms, for example, are also disclosed.

### BACKGROUND

Among the many food safety-related issues critical to public health, contamination caused by microorganisms and the various types of toxins they produce remains a major problem. Food-borne diseases caused by microbial contamination are among the world's most prominent food safety issues. Rapid and accurate assays for trace pathogenic microorganisms that may be present in food is a major operational need not only of sanitary and anti-epidemic departments but also of product quality control efforts by food manufacturers.

Traditional detection methods for trace pathogenic microorganisms require amplification or enrichment of target microorganisms in a sample in processes that are tedious, laborious, and time consuming. For example, culture isolation and identification methods have complex protocols, long detection periods, and often require special, specific culture and experimental conditions that must be precisely provided, as well as a high degree of technical expertise among operators. PCR and ELISA techniques have also been applied to the detection of pathogens. However, PCR positive results often require further confirmation by sequence determination. ELISA reactions are troubled by cross reaction to antibodies and challenges and costs associated with preserving protein samples. The application of real-time fluorescent PCR technology, for example, greatly improves detection accuracy. However, due to limitations in fluorescent label types and instrumental detection channels, for example, PCR detection is still cumbersome, time consuming and expensive.

### SUMMARY OF THE INVENTION

In one embodiment, a composite material is described that is made up of at least one quantum dot covalently bound to at least one prolamin protein such as a zein protein. In some examples, the bond may be formed from a carboxyl moiety or an amino group on the quantum dot surface. The quantum dot may be made of cadmium telluride, cadmium selenium, zinc sulfur, lead selenium, or rare earth doped colloidal phosphor nanoparticles. In one embodiment, quantum dot may be made of cadmium telluride. In some examples, the composite material also involves an antibody, which may be bound to the protein and may be configured to bind to a microorganism.

In one embodiment, the composite material is a film made of a plurality of quantum dots each covalently bound to at least one of a plurality of zein proteins. The film may be applied to a support structure, which may involve a receptacle for holding a sample, for example. The composite material may involve an antibody, which may be covalently bound to the protein. In some embodiments, this antibody may be configured to bind to a microorganism. In some embodiments, the film may be applied to a support structure.

In an example, a film is made of a plurality of quantum dots, a plurality of proteins and at least one antibody. The film may be applied to a support structure. In some embodiments, at least one antibody is bound to at least one of the proteins. In some embodiments the antibody is configured to bind to a microorganism. In some embodiments the plurality of proteins are prolamin proteins such as zein proteins.

In one embodiment, a method is disclosed for labeling at least one microorganism, including contacting the microorganism with a film that is made of quantum dots bound to proteins and at least one antibody. In some embodiments, the microorganism is able to degrade the protein. In some embodiments, the labeling involves at least one quantum dot being internalized within the microorganism. In some embodiments, an exogenous enzyme is added to degrade the protein constituent of the film.

In one embodiment, a method is taught for assaying for the presence of a microorganism in a sample. In some embodiments, the method includes contacting the sample with a film including at least one quantum dot bound to at least one protein and further including at least one antibody, where the antibody binds the microorganism; and visualizing the quantum dots. In some embodiments, the method involves contacting the composition with a reagent that degrades the at least one protein after contacting the sample with the film. In some embodiments, the visualization involves determining the emission spectrum of the quantum dot. In some embodiments, the microorganism is a food-borne pathogen. In some embodiments, the film is applied to the inner surface of a receptacle capable of containing a sample.

In one embodiment, a method for labeling a substrate is disclosed. The method may involve contacting more than one quantum dot to more than one protein; assembling the quantum dots into at least one multimer; attaching at least one antibody to the multimer to form a multimer-antibody complex; contacting a sample to be assayed with the multimer-antibody complex; and assaying for the presence of a signal from the quantum dot indicative of a microorganism in the sample. In some embodiments, the binding of the antibody to the microorganism causes the quantum dots of the multimer to be bound to the microorganism. In various embodiments, the multimer may be a film, and the protein may be a prolamin protein, such as zein. In some embodiments, the quantum dots are covalently bound to the proteins, and the covalent bond can be formed from at least one carboxyl moiety or amino moiety on the surface of the quantum dot. In some embodiments, the signal generated upon binding to a substrate is substantially greater than a signal generated when a similar sample is contacted with a comparable number of quantum-dot protein antibody complexes wherein the comparable complexes do not form multimers.

In one embodiment, a kit is taught for the detection of an epitope. The kit may include a film made in part of quantum dots bound to proteins and at least one antibody. The antibody may be configured to bind a microorganism such as a food-borne pathogen.

In one embodiment, a method of assaying for the presence of a food borne pathogen is taught. The method may include allowing at least one quantum dot to become associated with at least one protein to form at least one quantum-dot-protein complex; allowing the quantum-dot-protein complex to become associated with another quantum-dot-protein complex to form a quantum-dot-protein complex multimer; and allowing an antibody to become associated with at least one protein of the multimer. In some embodiments, the protein is a prolamin protein such as zein. In some embodiments, the quantum dots are covalently bound to the protein, and this bond may be formed, for example, from at least one carboxyl moiety or at least one amino moiety on the surface of the quantum dot. In some embodiments, the antibody can bind at least one food borne pathogen, and the multimer is contacted to a sample. The multimer may be configured as a film. The film may be applied to a surface. In some embodiments, the surface is an interior surface of a receptacle configured to contain a sample to be assayed. The interior surface may contact the sample.

In one embodiment, an apparatus is described for the detection of a food-borne pathogen. The apparatus may include a receptacle for the retention of a sample, and in some embodiments, the interior surface of the receptacle is configured to contact the sample, where at least a portion of the interior surface may be coated with a film made of a plurality of proteins, a plurality of quantum dots, and at least one antibody. In some embodiments, the proteins are prolamin proteins, such as zein proteins. In some embodiments, the quantum dots are covalently bound to the proteins. The at least one antibody may be bound to the proteins, and in some embodiments, the at least one antibody is configured to bind a food borne pathogen.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail, in part through use of the accompanying drawings.

FIG. 1. In an illustrative depiction, Cd/Te quantum dots presenting carboxyl groups on their surfaces are shown. The coupling agent EDC is used to bind a plurality of these quantum dots to Zein proteins, forming a composite quantum dot protein complex.

FIG. 2. TEM image of an illustrative composite material of proteins and quantum dots.

FIG. 3. Fluorescent photographs of NIH3T3 cells treated with quantum dots (Left) or quantum dot protein composite particles (Right).

FIG. 4. Preparation of quantum dot films. In an illustrative depiction, quantum dot protein composites may be used to form a film to which an antibody or antibodies may be applied. This film is useful in the detection, for example, of a food-borne pathogen such as a bacterium in a sample contacted by the antibody-film complex.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

Highly selective, sensitive and rapid materials and methods are disclosed for detecting pathogenic microorganisms in food.

Disclosed herein are compositions including quantum dots bound to zein protein. In some embodiments, each composite contains multiple quantum dots and thus achieves strong signal amplification. These compositions may further include at least one antibody. These compositions may be configured to form a film. In some embodiments, the film comprises an antibody. Also disclosed herein are quantum-dot protein films that also include an antibody. The proteins may comprise zein proteins, for example, but other proteins are contemplated as well. Also disclosed herein is a method of labeling at least one microorganism. The method may include contacting the microorganism with a film. The film may include quantum dots bound to proteins and at least one antibody. Also disclosed herein is a method of assaying for the presence of a microorganism in a sample. Also disclosed herein is a method of labeling a substrate. Also disclosed is a kit for the detection of an epitope such as an antigen on the surface of a microorganism such as a food-borne pathogen. Also disclosed is a method of assaying for the presence of at least one food borne pathogen. Also disclosed is an apparatus for the detection of a food-borne pathogen.

A quantum dot is a semiconductor material whose electron excitations are confined in all three spatial dimensions. Quantum dots have electron excitation properties intermediate between those of individual molecules and semiconductors. The excitation and emission properties of a given quantum dot are a function of the dot's size and shape. As a result, quantum dots can be designed to have specific absorption and emission spectra. This ability to tune the qualities of a quantum dot is very useful for a number of applications.

Quantum dots have uses in a broad range of applications, such as transistors, solar cells, LEDs, diode lasers, biological imaging, and have been explored for use in quantum computing. Quantum dots are attractive in computing because the flow of electrons through leads to quantum dots can be precisely controlled and measured.

Quantum dots are increasingly being used in biological imaging. They may be synthesized to cover a broad range of excitation and emission spectra. Also, their photostability and brightness are much improved relative to traditional imaging tools. These properties allow the acquisition of consecutive focal-plane images that can be reconstructed into a three-dimensional view of a cell. Quantum dots can be targeted to specific proteins through the use of antibodies, streptavidin, peptides, nucleic acid aptamers or small-molecule ligands.

A traditional example of a quantum dot used in immunodetection may include a quantum dot bound to a protein which is in turn bound to an antibody, having no higher-order structure. Traditional quantum dot-protein complexes do not provide sufficient signal or appropriate protein identity to allow some downstream applications.

### DEFINITIONS

As used herein, a “prolamin protein” refers to any member of the prolamine protein family, which includes a group of plant storage proteins found in the seeds of cereal grains. A nonlimiting list of prolamins includes gliadin from wheat, hordein from barley, secalin from rye, zein from corn, kafirin from sorghum and as a minor protein, avenin in oats. Prolamin homologues from other plant species are also contemplated. Sequences of prolamin proteins are known or readily available to those of skill in the art.

As used herein, “zein protein” refers to any of the prolamin proteins found in maize, including alpha-zeins, beta-zeins, gamma-zeins and delta-zeins. Many zeins are encoded by a large multi-gene family including multiple paralogous loci. Many zeins are unusually rich in glutamine, proline, alanine, and leucine residues. Sequences of zein proteins are known or readily available to those of skill in the art.

As used herein, “carboxyl” refers to “—C(═O)OH” group. As would be appreciated by the skilled artisan, a carboxyl group also includes its conjugate base.

As used herein, “amine” refers to “—NH” group. As would be appreciated by the skilled artisan, an amine group also includes its conjugate base.

As used herein, a “microorganism” refers to any organism a single representative of which is not visible to the naked eye. Examples include bacteria, most unicellular eukaryotes and small multicellular organisms. All steps in an organism's life cycle are contemplated so that, for example, microscopic eggs, spores or zygotes deposited by or capable of growing to become macroscopic organisms are contemplated by the use of ‘microorganism’ in this disclosure. Virus particles are similarly included in the definition of the term.

As discussed herein, “food-borne pathogen” refers to any organism which sickens, is capable of sickening, leads to a disease in, is capable of leading to a disease in, develops into a parasite of, is capable of developing into a parasite of, or otherwise negatively affects or is capable of negatively affecting a human, mammal or other animal consuming said food-borne pathogen.

### Protein-Conjugated Quantum Dots

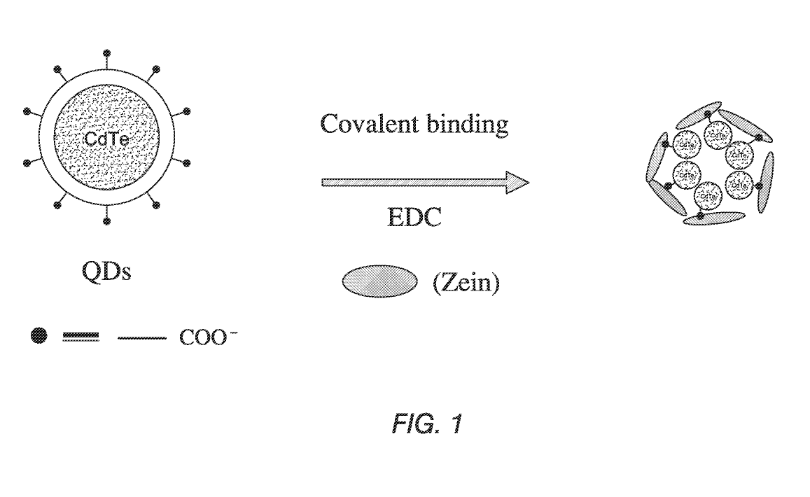

Some embodiments disclosed herein include quantum dots having an inorganic core and a protein shell. The core may include any number of quantum dots, such as cadmium telluride, cadmium selenium, zinc sulfur, lead selenium, and rare earth doped colloidal phosphor nanoparticles. Other quantum dot cores are also contemplated. FIG. 1 shows one example of a quantum dot composition that is within the scope of the present application. The quantum dot of FIG. 1 includes an inorganic core cadmium telluride having a surface of carboxyl groups, to which are bound an outer layer including zein protein. The outer layer may cover substantially all or all of the surface of inorganic core as shown in FIG. 1. In some embodiments, the shell may cover only a portion of the surface of the inorganic core (not shown).

The inorganic core in the quantum dot is not particularly limited and can be selected based on the desired properties. In some embodiments, the inorganic core can be conjugated to a protein in the shell.

The core can be selected from a number of well-known components. For example, the inorganic core can include a metal element. The metal element can be from main group II, subgroup VIIA, subgroup VIIIA, subgroup IB, subgroup IIB, main group III or main group IV of the periodic table. Examples of these elements include gold, silver, copper, titanium, terbium, cobalt, platinum, rhodium, ruthenium, and lead, although other examples are contemplated. The inorganic core may include a single pure metal, or an alloy of two, three, or more than three metals. The alloy may include any of the metals disclosed in the present application, or may include other elements known by one of skill in the art.

The inorganic core can include a semiconductor. For example, the semiconductor may include a metal from main group II or subgroup IIB and an element from main group VI. Alternately, the semiconductor may include a metal from main group III and an element from main group V. Some well-known examples of semiconductors include, but are not limited to, AIN, AlP, AlAs, AlSb, CdS, CdSe, CdTe, GaAs, GaN, GaP, GaSb, HgS, HgSe, HgTe, InAs, InN, InP, InSb, MgTe, ZnS, ZnSe, and ZnTe.

The core can include an oxide. Non-limiting examples of oxides include silicon dioxide (SiO₂), and metal oxides such as aluminum oxide (Al₂O₃), titanium dioxide (TiO₂) and zirconium dioxide (ZrO₂).

The size of the inorganic core is also not particularly limited. The inorganic core may, for example, have an average diameter of no more than about 30 nm; no more than about 20 nm; no more than about 15 nm; or no more than about 10 nm. The inorganic core may, for example, have an average diameter of at least about 1 nm; at least about 2 nm; at least about 3 nm; at least about 5 nm; at least about 7 nm; at least about 10 nm; or at least about 15 nm. The inorganic core may also have a diameter between any of these values. For example, the inorganic core can have an average diameter of about 1 nm to about 15 nm.

The inorganic core may be prepared using standard methods known in the art. For example, the inorganic core may be prepared by injecting organometallic precursors into a hot coordinating solvent, as described in U.S. Publication No. 2004/0033359.

The shell may include a prolamin such as zein. A nonlimiting list of prolamins includes gliadin from wheat, hordein from barley, secalin from rye, zein from corn, kafirin from sorghum and as a minor protein, avenin in oats. Prolamin homologues from other plant species are also contemplated. Zein refers to any of the prolamin proteins found in maize, including alpha-zeins, beta-zeins, gamma-zeins and delta-zeins. Many zeins are encoded by a large multi-gene family including multiple paralogous loci. Many zeins are unusually rich in glutamine, proline, alanine, and leucine residues, but the term as used herein is not limited by this trait.

In some embodiments, zein protein is selected because it includes a high quantity of hydrophobic amino acids which have beneficial properties such as the following: good film-forming property, adhesion, and resistance to water, acid and oil. Therefore, protein-conjugated quantum dots including zein have a high hydrophobicity such that they can be easily prepared into film which has a strong binding force with a substrate material, thereby providing the basis for detection. Additionally, zein proteins are abundant in nature, easily acquired and have a relatively low market price.

In some embodiments, the protein-conjugated quantum dots may possess unique properties, such as strong light-emitting signals, excellent light stability, and ease of coupling with biological molecules for use in biological interaction and recognition. Nanoparticles modified by antibodies may bind to corresponding antigens on the surface of the microorganisms and detect microorganisms with a high selectivity. In some embodiments, microorganisms can bind with a plurality of protein-conjugated quantum dots, facilitating detection of the microorganisms.

In some embodiments, the surfaces of the protein-conjugated quantum dots can be easily modified to endow them with certain charges and functional groups, such that modification of the protein-conjugated quantum dots with specific antibodies against different pathogenic microorganisms can identify and detect a variety of sources of microorganisms such as food-borne pathogens.

### Methods of Synthesizing Protein-Conjugated Quantum Dots

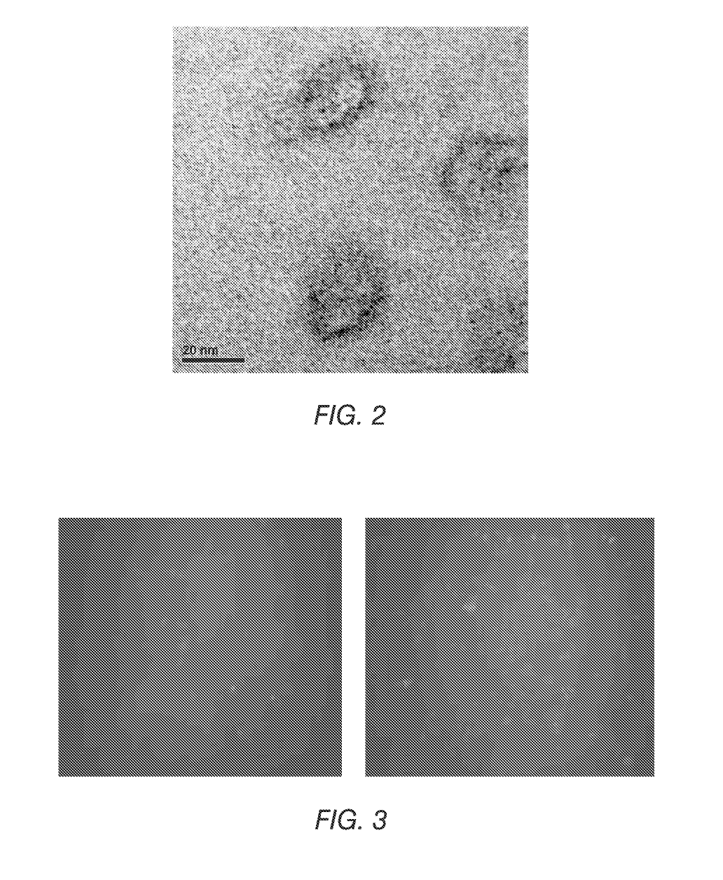

Protein-conjugated quantum dots can be synthesized using a number of methods known to one of skill in the art. As a nonlimiting example, proteins such as zein can be conjugated to quantum dots using the coupling agent 1-ethyl-3-(3-dimethylaminopropyl) carbodiimide) (“EDC”), as described by Mee Hyang Ko, et al. (In vitro Derby imaging of cancer biomarkers using quantum dots. Small 2009, 5(10):1207-1212). FIG. 1 depicts one such synthesis method. Through this method, zein protein is covalently bound to a carboxyl moiety on the surface of the quantum dot. This method may also be used to bind proteins to amino moieties on a quantum dot surface. Carboxyl and amino-modified quantum dots are commercially available from reagent companies that sell quantum dots. FIG. 2 depicts zein-conjugated quantum dots synthesized through such method. Other cross-linkers contemplated include 1-cyclohexyl-3-(2-morpholinoethyl) carbodiimide (“CMC”) and dicyclohexyl carbodiimide (“DCC”), for example.

### Protein-Conjugated Quantum Dot Films

In some embodiments, the protein-conjugated quantum dot complexes above can be assembled into multimers such as films to which one or more antibodies may be bound. Antibodies may be bound to the quantum dots through any of a number of methods known to one of skill in the art. For example, antibodies may be bound to the proteins covalently, using cross-linkers such as those mentioned above. Alternately, a binding system such as the Biotin-Avidin system may be used to bind an antibody to the protein-conjugated quantum dot complexes. Amplification of light-emitting signals of the quantum dots may be effected through the presence of multiple protein-conjugated quantum dot complexes within a single film. In some embodiments this arrangement results in strong optical signal amplification, such that improved detection capabilities can be achieved. FIG. 3 depicts the improved detection capabilities that may be achieved in some embodiments of protein-conjugated quantum dot complex antibody films.

In some embodiments, these films may be applied to the surface of receptacles capable of holding samples to be assayed. In some embodiments multi-well apparatuses may be used to effect high-throughput screening of samples for, for example, food-borne pathogens bound by the attached antibodies.

In some embodiments, upon binding to a target microorganism such as a food-borne pathogen or other microorganism, the non-antibody protein constituent of the protein-conjugated quantum dot film may be degraded. This degradation may be effected by, for example, microbial enzymes or exogenous enzymes, and the quantum dots may enter into the microorganism. Entry of the quantum dots into the microorganism may be an active or passive process. That is, in some embodiments the microorganism takes up the bound protein-conjugated quantum dot and degrades the protein constituent to release the quantum dots, in some embodiments the microorganism secretes a substance that degrades the non-antibody protein constituent of the protein-conjugated quantum dot film, and in some embodiments an externally supplied substance degrades or triggers degradation of the non-antibody protein constituent of the protein-conjugated quantum dot film. In some embodiments this entry may result in a microorganism being internally labeled with the quantum dots of the protein-conjugated quantum dot film. In some embodiments an exogenous enzyme is added after the microorganism has taken up the bound protein-conjugated quantum dot or dots, and in some embodiments this exogenous enzyme may facilitate the dispersal of the quantum dot labeled microorganism from the film. In some embodiments dispersal of the labeled microorganism may facilitate later detection, for example through flow cytometry.

### Synthesis of Protein-Conjugated Quantum Dot Films

In some embodiments the film-forming properties of a prolamin protein such as zein are used to prepare protein-conjugated quantum dot films. Protein-conjugated quantum dots including zein have a high hydrophobicity such that they can be easily prepared into film which has a strong binding force with a substrate material, thereby providing the basis for detection. In some embodiments these films are applied to a substrate such as a sample receptacle. In some embodiments, one or more antibodies may be bound to the film. Upon binding a microorganism through specific antibodies, the interaction between the microorganism and films can cause changes in fluorescent properties (wavelength, intensity or distribution of emissions), and a fluorescence detection system may detect these changes so as to achieve the purpose of detection of microorganisms in a sample.

### Method of Using Protein-Conjugated Quantum Dots and Films

Florescence microscopy may be used to visualize quantum dot localization. Some embodiments disclosed herein relate to a method of using changes in light emission spectra, intensity or spatial distribution to indicate the presence of a microorganism. The method can involve exposing a composition having quantum dots to electromagnetic radiation that is effective to excite the quantum dot electrons. The quantum dot can be any of those disclosed in the present application, and the excitation energy will vary among quantum dots selected, but will be apparent to one of skill in the art. In some embodiments, the method further includes detecting the luminescence emitted by the quantum dot in the form of an emission spectrum.

In some embodiments, the method further includes detecting the luminescence intensity, wavelength or spatial distribution produced from the quantum dot and correlating the luminescence intensity, wavelength or spatial distribution with a concentration, chemical status or spatial distribution of quantum dots in the composition.

The quantum dot can be any of those disclosed in the present application, and can be individual or configured in a multimer such as a film. An increased intensity, a shifted emission wavelength, or a change in the spatial distribution of quantum dot emission spectra, for example, may indicate the presence of an antigen to be detected such as a microorganism in a sample.

In some embodiments, the system can be a sample, such as a food or drink sample. The method can be used, for example, to detect microorganisms in the sample.

In some embodiments, an antibody having affinity for an antigen is bound to the quantum dot-protein composition. In some embodiments, the antigen is an epitope. In some embodiments, the antigen is on the surface of a microorganism such as a food-borne pathogen.

In some embodiments, regions exhibiting increased luminescence intensity at the emission wavelength can be correlated with the presence of a microorganism, for example, in a sample. In some embodiments, a change in spatial distribution of the emission may indicate the presence of a microorganism to be assayed.

Florescence emitted by a quantum dot may be detected using florescence microscopy, high-throughput automated plate florescence readers, flow cytometry, or other techniques which are similarly well-known to one of skill in the art.

An assay for, for example, a microorganism in a sample may be effected by contacting a sample to a protein-conjugated quantum dot, a protein-conjugated quantum dot film, or to a receptacle to which a protein-conjugated quantum dot film has been applied. Quantum dots may be excited using electromagnetic waves of the appropriate excitation wavelength, which may vary among quantum dots but will be known to one skilled in the art, and their emission spectra may be detected as above.

### Apparatus to Detect Food-Borne Pathogens

The method of assaying for microorganisms such as food borne-pathogens discussed above may be practiced using a receptacle to contain one or more samples to be assayed. Such a receptacle may be coated with an antibody-bound protein-conjugated quantum dot film on a surface that comes into contact with a sample to be assayed. In some embodiments the apparatus is partially or substantially transparent to the excitation and emission spectra of the quantum dot used. In some embodiments the apparatus may include multiple receptacles such that a plurality of samples may be contained within a single apparatus simultaneously without substantial mixing of samples. In some embodiments the apparatus is configured to match a device capable of generating electromagnetic energy at the appropriate excitation wavelength and detecting energy at the appropriate emission wavelength such that multiple samples may be assayed simultaneously or in rapid succession.

### EXAMPLES

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

### Example 1

### Conjugation of Zein to Cd/Te Quantum Dots

Cadmium/Telluride quantum dots presenting carboxyl groups on their surface were contacted with zein proteins in the presence of 1-ethyl-3-(3-dimethylaminopropyl) carbodiimide) (“EDC”). EDC activates the carboxyl agents of the quantum dot surface, then bind primary amines of the zein protein. The reaction is performed under conditions sufficient to yield zein conjugated Cd/Te quantum dots including multiple zein proteins and multiple Cd/Te quantum dots in each complex. See FIG. 1. The reaction yielded spherical complexes with diameter of approximately 20 nm, shown in FIG. 2.

### Example 2

### Improvement in Detection Capability Through the Use of Multiple Quantum Dotes Per Antibody Complex

Zein conjugated Cd/Te quantum dots including multiple zein proteins and multiple Cd/Te quantum dots in each complex were bound to NIH3T3 cells. Said cells were contacted with either solitary quantum dots (as shown in FIG. 3, Left panel) or Zein conjugated Cd/Te quantum dots including multiple zein proteins and multiple Cd/Te quantum dots in each complex (as shown in FIG. 3, Right). The results indicate an improved visualization of the target cells using Zein conjugated Cd/Te quantum dots including multiple zein proteins and multiple Cd/Te quantum dots in each complex.

### Example 3

### Zein Films in Food-Borne Pathogen Detection

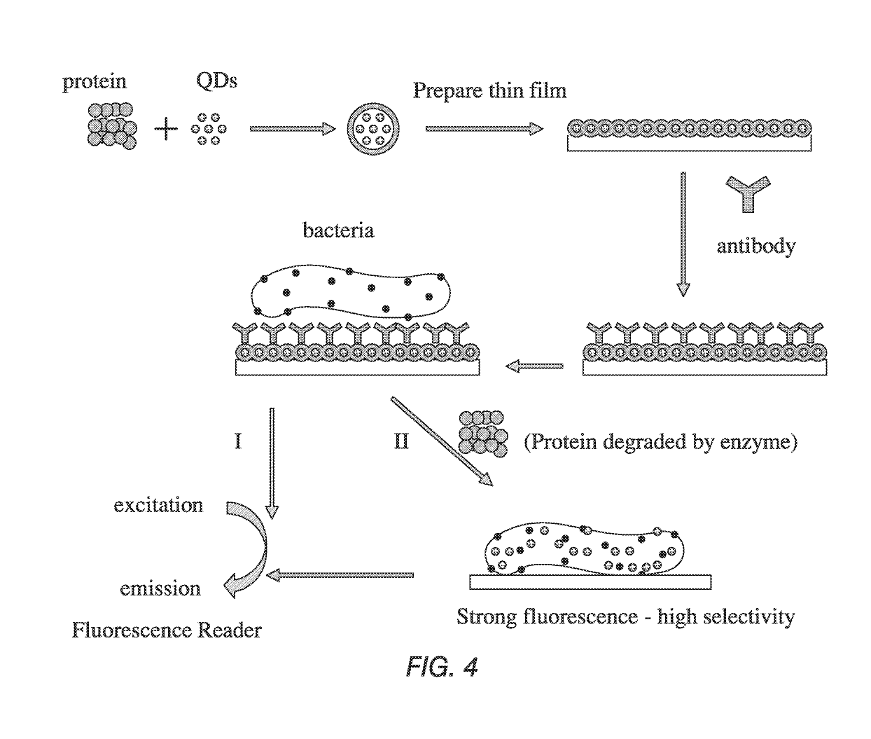

Zein conjugated Cd/Te quantum dots of Example 1 were applied to a substrate to form a film, making use of the chemical properties of zein which facilitate this formation. Antibodies specific to a bacterial food-borne pathogen were bound to the film thus formed. The antibody-bound film was contacted by a bacterial food-borne pathogen presenting an epitope bound by the antibody. The bacterium so bound was detected by observing an altered emission spectrum distribution characteristic of binding. The process by which quantum dot films are made and used to improve detection sensitivity is shown in FIG. 4.

### Example 4

### Degradation of Substrate-Bound Zein Films by Microbes Facilitates Detection of a Food-Borne Pathogen

Films were prepared and contacted to a food-borne bacterial pathogen as in Example 3. Following binding of the bacterium, Zein conjugated Cd/Te quantum dots were internalized and the protein constituent was degraded, freeing the Cd/Te quantum dots. This change in the distribution of quantum dots, indicated by a change in the distribution of their emission spectra, indicated the presence of a bacterium of the type assayed in the sample. The process by which uptake of quantum dots by a bacterium is used to improve detection sensitivity is shown in FIG. 4.

### Example 5

### Treatment of Substrate-Bound Zein Films with a Zein-Degrading Enzyme to Facilitate Detection of a Food-Borne Pathogen

Films were prepared and contacted to a food-borne bacterial pathogen as in Example 3. Following binding of the bacterium and internalization of the quantum dots, the film was treated with an enzyme that degraded the zein protein, freeing the microorganism from the film. This change in the distribution of quantum dots, indicated by a change in the distribution of their emission spectra, indicated the presence of a bacterium of the type assayed in the sample. The process by which a zein-degrading enzyme is used to improve detection sensitivity is shown in FIG. 4.

### Example 6

### Detection of a Labeled Food-Borne Pathogen

The labeled, enzyme-treated microorganism of Example 5 is allowed to disperse from the quantum dot film. The localization of dots at the microorganism is then detected in a flow cytometer.

### General Comments

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

While the present invention has been described in some detail for purposes of clarity and understanding, one skilled in the art will appreciate that various changes in form and detail can be made without departing from the true scope of the invention.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A composite material, comprising at least one quantum dot covalently bound to at least one prolamin protein, wherein the covalent bond is formed by the reaction of at least one carboxyl moiety or amino group on the surface of the quantum dot.

2. The composite material of claim 1, wherein the prolamin protein is a zein protein.

3. (canceled)

4. The composite material of claim 1, wherein the quantum dot comprises a composition selected from the group comprising cadmium telluride, cadmium selenium, zinc sulfur, lead selenium, and rare earth doped colloidal phosphor nanoparticles.

5. The composite material of claim 1, wherein the quantum dot comprises cadmium telluride.

6. The composite material claim 1, further comprising at least one antibody.

7. The composite material of claim 6, wherein the antibody is bound to the protein.

8. The composite material of claim 6, wherein the antibody is configured to bind to a microorganism.

9. A film comprising a plurality of quantum dots each covalently bound to at least one prolamin protein.

10. The film of claim 9, wherein the protein is a prolamin protein.

11. The film of claim 9, wherein the protein is a zein protein.

12. The film of claim 9, wherein the film is applied to a support structure.

13. The film of claim 12, wherein the support structure comprises a receptacle for holding a sample.

14. The film of claim 9, further comprising at least one antibody.

15. The film of claim 14, wherein the at least one antibody is bound to the protein.

16. The film of claim 15, wherein the antibody is configured to bind to a microorganism.

17-20. (canceled)

21. A method of assaying for the presence of a food borne pathogen in a sample, the method comprising:
contacting the sample with a film comprising a plurality of quantum dots bound to at least one protein and further comprising at least one antibody configured to bind the food borne pathogen, wherein the quantum dots are configured to generate a detectable signal when the at least one antibody is bound to the food borne pathogen; and
detecting the signal from the quantum dots.

22-24. (canceled)

25. The method of claim 21, further comprising applying the film to an inner surface of a receptacle capable of containing the sample.

26-44. (canceled)

45. An apparatus for the detection of a food-borne pathogen comprising a receptacle for the retention of a sample, wherein an interior surface of the receptacle is coated with a film comprising a plurality of proteins, a plurality of quantum dots, and at least one antibody.

46. The apparatus of claim 45, wherein the plurality of proteins comprise prolamin proteins.

47. The apparatus of claim 46, wherein the prolamin proteins are zein proteins.

48. The apparatus of claim 45, wherein the quantum dots are covalently bound to the proteins.

49. The apparatus of claim 45, wherein the at least one antibody is covalently bound to the proteins.

50-51. (canceled)

52. The method of claim 21, wherein contacting results in at least one quantum dot being internalized within the food borne pathogen.

53. The method of claim 21, further comprising treating the sample with an enzyme that degrades the protein.
